(19) United States
(12) Patent Application Publication
(10) Pub. No.: US 2026/0248488 A1
(43) Pub. Date: Aug. 27, 2026

(54) ARRHYTHMIA TYPE INFERENCE DEVICE, ARRHYTHMIA TYPE INFERENCE METHOD, AND RECORDING MEDIUM

(71) Applicants: KANADEVIA CORPORATION, Osaka (JP); NATIONAL CEREBRAL AND CARDIOVASCULAR CENTER, Osaka (JP)

(72) Inventors: Jun YOSHIMATSU, Osaka (JP); Aiko KAKIGANO, Osaka (JP); Ryo ITOH, Osaka (JP); Hiroki MATSUZAKI, Osaka (JP); Masahiro TOMARU, Osaka (JP)

(21) Appl. No.: 19/551,827

(22) Filed: Feb. 27, 2026

(30) Foreign Application Priority Data
Feb. 27, 2025 (JP) 2025-030305

(51) Int. Cl. A61B 8/00; A61B 8/08; G06T 7/62

(52) U.S. Cl. CPC A61B 8/5223; A61B 8/0883; G06T 7/62; G06T 2207/10132; G06T 2207/30048

(57) ABSTRACT

An arrhythmia type inference device includes: an obtaining section that obtains a target signal waveform indicating a time-series change in the area of a region, which is at least one of a left atrium, a left ventricle, a right atrium, and a right ventricle, the target signal waveform being generated by analyzing an image of a heart of a target subject; and an inference section that infers the type of tachyarrhythmia suffered by the target subject based on the shape of the target signal waveform having a frequency in a frequency band higher than a reference frequency band predetermined as a normal range. This allows the arrhythmia type inference device to accurately infer the type of tachyarrhythmia from the image obtained by image-capturing of the heart.

This Nonprovisional application claims priority under 35 U.S.C. §119 on Patent Application No. 2025-030305 filed in Japan on Feb. 27, 2025, the entire contents of which are hereby incorporated by reference.

### TECHNICAL FIELD

The present invention relates to a technique for determining the type of arrhythmia.

### BACKGROUND ART

There is a widely-used technology for grasping a relation between the left and right sides of the heart and a relation between atria and ventricles from a moving image of a heart obtained by ultrasonography. For example, Patent Literature 1 discloses an ultrasound diagnostic device that identifies a group of boundary positions between a plurality of heart chambers in a moving image obtained by ultrasonography and obtains, on the basis of a result of tracking the group of the boundary positions, the boundary positions between the plurality of heart chambers over a period of at least one heartbeat.

### Citation List

### Patent Literature

Patent Literature 1

Japanese Patent Application Publication, Tokukai, No. 2022-149097

### SUMMARY OF INVENTION

### Technical Problem

Conventionally, electrocardiograms are mainly used for diagnosing arrhythmia. However, it may be difficult to determine the type of tachyarrhythmia that has occurred, and there has been room for improvement in terms of determination accuracy.

An object of an aspect of the present invention is to accurately infer the type of tachyarrhythmia from an image obtained by image-capturing of a heart.

### Solution to Problem

An arrhythmia type inference device according to an aspect of the present invention includes: an obtaining section that obtains a target signal waveform indicating a time-series change in an area of a region, which is at least one of a left atrium, a left ventricle, a right atrium, and a right ventricle, the target signal waveform being generated by analyzing an image of a heart of a target subject; and an inference section that infers the type of tachyarrhythmia suffered by the target subject, based on a shape of the target signal waveform having a frequency in a frequency band higher than a reference frequency band predetermined as a normal range.

An arrhythmia type inference method according to an aspect of the present invention is an arrhythmia type inference method performed by one or more information processing devices, said method including: an obtaining step of obtaining a target signal waveform indicating a time-series change in an area of a region, which is at least one of a left atrium, a left ventricle, a right atrium, and a right ventricle, the target signal waveform being generated by analyzing an image of a heart of a target subject; and an inference step of inferring the type of tachyarrhythmia suffered by the target subject, based on a shape of the target signal waveform having a frequency in a frequency band higher than a reference frequency band predetermined as a normal range.

The arrhythmia type inference device in accordance with the foregoing aspects of the present invention may be achieved by a computer. In such a case, the present invention encompasses: a control program for the arrhythmia type inference device that causes a computer to operate as each of the sections (software elements) of the arrhythmia type inference device so that the arrhythmia type inference device can be achieved by the computer; and a computer-readable storage medium storing the control program therein.

### Advantageous Effects of Invention

According to an aspect of the present invention, it is possible to accurately infer the type of tachyarrhythmia from an image obtained by image-capturing of a heart.

### BRIEF DESCRIPTION OF DRAWINGS

FIG. 1 is a diagram showing a configuration example of an arrhythmia type inference system according to an embodiment of the present invention.

FIG. 2 is a view showing an example in which respective regions of a left atrium, a left ventricle, a right atrium, and a right ventricle are detected from an ultrasound image of a heart.

FIG. 3 is a block diagram showing an example of the configuration of the main parts of an arrhythmia type inference device according to Embodiment 1 of the present invention.

FIG. 4 is a diagram for explaining an example of processing performed by an inference section.

FIG. 5 is a flowchart showing an example of a flow of processing performed by the arrhythmia type inference device.

FIG. 6 is a block diagram showing an example of the configuration of the main parts of an arrhythmia type inference device according to Embodiment 2 of the present invention.

FIG. 7 is a diagram for explaining an example of processing performed by a single-beat waveform obtaining section.

FIG. 8 is a diagram for explaining an example of processing performed by the single-beat waveform obtaining section.

FIG. 9 is a diagram for explaining an example of a first preprocessing.

FIG. 10 is a diagram for explaining an example of processing performed by an inference section.

FIG. 11 is a flowchart showing an example of a flow of processing performed by the arrhythmia type inference device.

FIG. 12 is a block diagram showing an example of the configuration of the main parts of an arrhythmia type inference device according to Embodiment 3 of the present invention.

FIG. 13 is a diagram for explaining an example of processing performed by a second preprocessing section and an inference section.

FIG. 14 is a flowchart showing an example of a flow of processing performed by the arrhythmia type inference device.

### DESCRIPTION OF EMBODIMENTS

### Embodiment 1

The following description will discuss the details of an embodiment of the present invention.

### Overview of arrhythmia type inference device 1

An arrhythmia type inference device 1 according to an embodiment of the present invention infers the type of tachyarrhythmia suffered by a target subject, based on the shape of a target signal waveform generated by analyzing an image of a heart of the target subject. It should be noted here that the target signal waveform is data indicating a time-series change in the area of a region, which is at least one of a left atrium, a left ventricle, a right atrium, and a right ventricle, in the image of the heart of the target subject. Hereinafter, the area of each region corresponding to the left atrium, the left ventricle, the right atrium, and the right ventricle is referred to as "area of each region".

The inventors have found that the shape of a signal waveform indicating a time-series change in the area of a region, which is at least one of the left atrium, the left ventricle, the right atrium, and the right ventricle included in an image obtained by image-capturing of a heart, exhibits characteristics corresponding to the type of tachyarrhythmia occurring in the heart. The arrhythmia type inference device 1 is capable of accurately inferring the type of tachyarrhythmia suffered by the target subject, from the image obtained by image-capturing of the heart of the target subject.

The tachyarrhythmia is an arrhythmia having a frequency in a frequency band higher than a reference frequency band (for example, 100 bpm or more and 170 bpm or less) predetermined as a normal range. The tachyarrhythmia includes, for example, atrial flutter (hereinafter referred to as "AFL") and supraventricular tachycardia (hereinafter referred to as "SVT"). AFL is an arrhythmia in which fine movements of approximately 200 to 350 bpm occur in the atria, and SVT is an arrhythmia in which movements of the atria and ventricles become faster than the frequency of the reference frequency band.

The target subject may be an animal having a heart and a fetus thereof. For example, the target subject may be a human fetus. In this case, the area of each atrium and each ventricle may be calculated based on an ultrasound image including images of a plurality of frames obtained by image-capturing of the fetus. The heart of the fetus is captured in the ultrasound image obtained by image-capturing of the fetus, and the area of each region of the heart of the fetus can be calculated from the ultrasound image. Processing for calculating the area of each region of the heart from the ultrasound image will be described later with a specific example.

### Configuration of arrhythmia type inference system 100

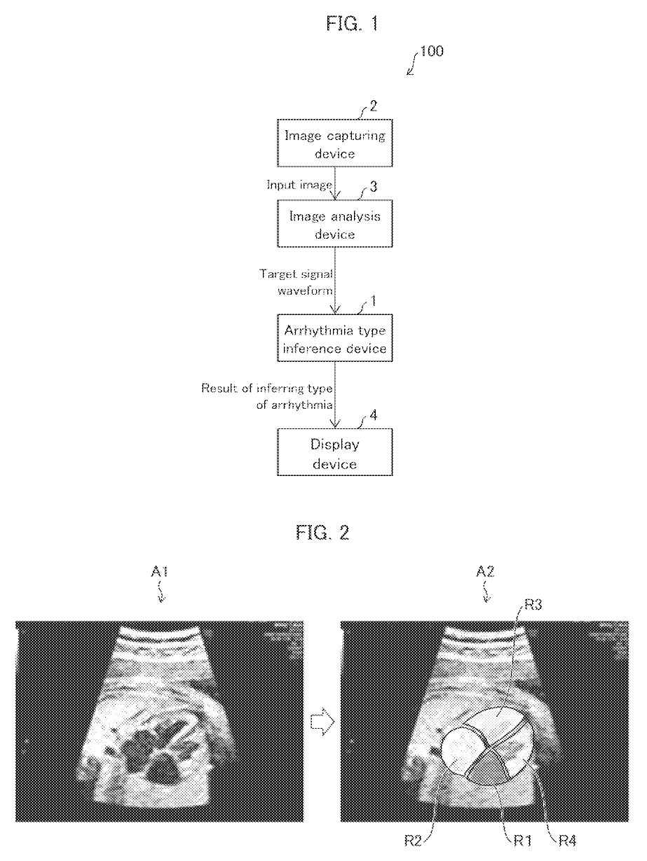

First, the configuration of an arrhythmia type inference system 100 including the arrhythmia type inference device 1 according to an embodiment of the present invention will be described with reference to FIG. 1. FIG. 1 is a diagram showing a configuration example of the arrhythmia type inference system 100. The arrhythmia type inference system 100 may include an image capturing device 2, an image analysis device 3, the arrhythmia type inference device 1, and a display device 4.

### Image capturing device 2

The image capturing device 2 may be an ultrasound image capturing device capable of non-invasively capturing an image of the inside of the body of the target subject. That is, an input image captured by the image capturing device 2 may be, for example, an ultrasound image such as an echo image for tomographic image formation. The image capturing device 2 may be communicably connected to the image analysis device 3 as illustrated, and in this case, the image analysis device 3 may directly obtain the input image from the image capturing device 2. It should be noted that the input image may be stored in an image management device (not shown) in association with target subject information (for example, electronic medical record information) for each target subject, and in this case, the arrhythmia type inference device 1 may obtain the input image from the image management device.

### Image analysis device 3

The image analysis device 3 detects respective regions of the left atrium, the left ventricle, the right atrium, and the right ventricle of the heart of the target subject captured in the input image. Furthermore, the image analysis device 3 calculates the area of each detected region and generates a target signal waveform indicating a time-series change in the area of each region.

The image analysis device 3 may be installed in a facility (for example, a medical facility) where the image capturing device 2 is installed, or may be installed in a remote place. When the image analysis device 3 is installed in a remote place, the image analysis device 3 may obtain the input image by communication via a communication network such as the Internet.

Processing for detecting each region corresponding to each atrium and each ventricle of the heart from the input image will be described with reference to FIG. 2. FIG. 2 is a view showing an example in which respective regions of a left atrium, a left ventricle, a right atrium, and a right ventricle are detected from an ultrasound image of a heart. An image A1 shown in FIG. 2 is an ultrasound image of the heart, and an image A2 shows detection results of respective regions R1 to R4 superimposed on the ultrasound image.

The heart is captured in a region slightly below the center in the image A1, and an outer shape thereof is visible, and it is also visible that the inside of the heart is divided into a plurality of sections. As described above, in the ultrasound image, each of two ventricles and each of two atria included in the heart can be visually recognized as closed regions. Therefore, by analyzing the ultrasound image of the heart, it is possible to detect the respective regions R1 to R4 of the left atrium, the left ventricle, the right atrium, and the right ventricle.

For example, machine learning can be performed using training data in which labels such as left atrium, left ventricle, right atrium, and right ventricle are attached as ground truth data to regions corresponding to the left atrium, the left ventricle, the right atrium, and the right ventricle of the heart captured in the ultrasound image of the heart. The label can also be referred to as an annotation. By such machine learning, a trained model capable of detecting the respective regions R1 to R4 of the left atrium, the left ventricle, the right atrium, and the right ventricle from the ultrasound image of the heart can be constructed. For example, by constructing a trained model of a convolutional neural network and using the trained model, highly accurate region detection becomes possible.

The image A2 shown in FIG. 2 shows a result of detection using such a trained model. The detected regions R1 to R4 are regions detected as the right ventricle, the left ventricle, the left atrium, and the right atrium, respectively. By detecting the respective regions R1 to R4, it becomes possible to calculate the area of each of the regions R1 to R4. For example, the area of each of the regions R1 to R4 can be represented by the number of pixels included in each of the detected regions R1 to R4. By calculating the area of each of the regions R1 to R4 of the left atrium, the left ventricle, the right atrium, and the right ventricle of the heart of the target subject in each frame of the ultrasound image, it is possible to obtain the target signal waveform indicating the time-series change in the area of each of the regions R1 to R4.

### Arrhythmia type inference device 1

The arrhythmia type inference device 1 infers the type of tachyarrhythmia suffered by the target subject by using the target signal waveform obtained from the image analysis device 3. According to the arrhythmia type inference device 1, it is possible to output a more highly accurate inference result as compared with a case where the type of tachyarrhythmia is inferred using an electrocardiogram.

The arrhythmia type inference device 1 may be communicably connected to the image analysis device 3 as illustrated, and in this case, the arrhythmia type inference device 1 may directly obtain the target signal waveform from the image analysis device 3. It should be noted that the target signal waveform generated by the image analysis device 3 may be stored in, for example, a portable recording medium, and in this case, the arrhythmia type inference device 1 may read the target signal waveform from the recording medium. Alternatively, the target signal waveform generated by the image analysis device 3 may be stored in, for example, any storage device (not shown) in association with the target subject information for each target subject, and in this case, the arrhythmia type inference device 1 may obtain the target signal waveform from the storage device.

The arrhythmia type inference device 1 may be installed in a facility (for example, a medical facility) where the image analysis device 3 is installed, or may be installed in a remote place. When the arrhythmia type inference device 1 is installed in a remote place, the arrhythmia type inference device 1 may obtain the target signal waveform generated by the image analysis device 3 by communication via a communication network such as the Internet.

A configuration may be adopted in which another computer executes a part of processing performed by the arrhythmia type inference device 1. That is, the processing performed by the arrhythmia type inference device 1 may be executed by one or more information processing devices. Alternatively, the arrhythmia type inference device 1 may have the function of the image analysis device 3. For example, when the arrhythmia type inference device 1 also has the function of the image analysis device 3, the image analysis device 3 is omitted from components of the arrhythmia type inference system 100.

### Display device 4

The display device 4 is a device capable of displaying various kinds of information output from the arrhythmia type inference device 1. The display device 4 may display the input image output from the image capturing device 2, the target signal waveform generated by the image analysis device 3, and the like, in addition to the various kinds of information output from the arrhythmia type inference device 1.

(Configuration of arrhythmia type inference device 1)

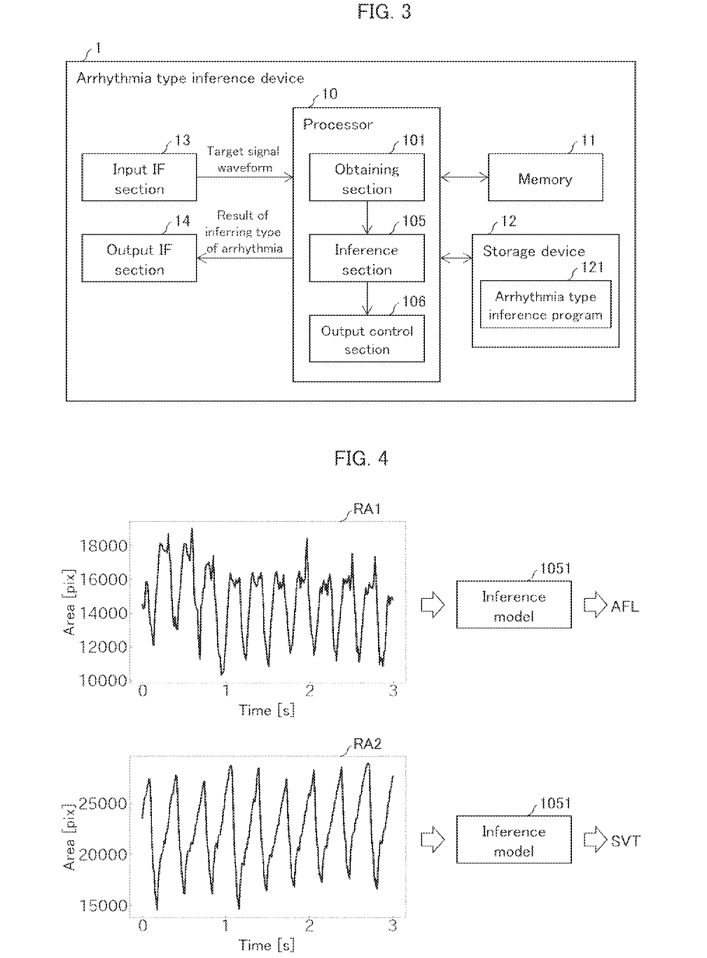

Subsequently, the configuration of the arrhythmia type inference device 1 according to an embodiment of the present invention will be described with reference to FIG. 3. FIG. 3 is a block diagram showing an example of the configuration of the main parts of the arrhythmia type inference device 1. The arrhythmia type inference device 1 having a function of inferring the type of tachyarrhythmia suffered by the target subject from the target signal waveform obtained from the image analysis device 3 will be described below as an example.

As illustrated, the arrhythmia type inference device 1 includes a processor 10, a memory 11, and a storage device 12. The arrhythmia type inference device 1 may be a personal computer, a server, or a workstation. The processor 10 functions as each section from an obtaining section 101 to an output control section 106 described later by loading an arrhythmia type inference program 121 stored in the storage device 12 into the memory 11 and executing the program.

The processor 10 can be achieved by a logic circuit (hardware) formed in an integrated circuit (IC chip) or the like, or can be achieved by software. When achieved by software, the processor 10 may be composed of, for example, a central processing unit (CPU), a graphics processing unit (GPU), or a combination thereof. In this case, the software is stored in the storage device 12. Then, the processor 10 reads the software into the memory 11 and executes the software.

Both the memory 11 and the storage device 12 are storage devices that store various kinds of data used by the arrhythmia type inference device 1. The memory 11 is a storage device capable of writing and reading data at a higher speed than the storage device 12. The storage device 12 has a larger data storage capacity than the memory 11. As the memory 11, for example, a high-speed access memory such as a synchronous dynamic random-access memory (SDRAM) can be applied. Furthermore, as the storage device 12, for example, a hard disk drive (HDD), a solid-state drive (SSD), a secure digital (SD) card, an embedded multi-media controller (eMMC), or the like can be applied.

Furthermore, the arrhythmia type inference device 1 includes an input IF section 13 and an output IF section 14 as interfaces (IF) with external devices. The input IF section 13 is an interface for accepting an input signal from an input device such as a keyboard or a mouse, or obtaining various kinds of information and data from an external device. The input IF section 13 is, for example, an interface for connecting the image analysis device 3 to the input IF section 13 and obtaining the target signal waveform and the like from the image analysis device 3. The output IF section 14 is an interface for outputting an inference result of the type of tachyarrhythmia and the like to an external device. The output IF section 14 can also, for example, connect a display device to the output IF section 14 and cause the display device to display the inference result of the type of tachyarrhythmia and the like.

The processor 10 functions as each of the obtaining section 101 that obtains the target signal waveform from the image analysis device 3, an inference section 105, and the output control section 106 by executing the arrhythmia type inference program 121.

It should be noted that the processor 10 may further include a selection section (not shown) that selects, from among the target signal waveforms obtained from the image analysis device 3, a target signal waveform in which a pulse has a frequency higher than a reference frequency band predetermined as a normal range. In this case, the obtaining section 101 obtains the target signal waveform selected by the selection section. Alternatively, a configuration may be adopted in which the obtaining section 101 (or the inference section 105) has the function of the selection section, instead of the configuration in which the processor 10 includes the selection section.

The inference section 105 infers the type of tachyarrhythmia suffered by the target subject based on the shape of the target signal waveform obtained by the obtaining section 101.

The inference section 105 may be configured to infer the type of tachyarrhythmia suffered by the target subject from the target signal waveform using a trained inference model 1051. As an example, the inference model 1051 may be machine-learned using training data in which an input waveform generated using a sample signal waveform generated by analyzing images of hearts of a plurality of sample subjects suffering from tachyarrhythmia is used as an explanatory variable and in which type information indicating the type of tachyarrhythmia suffered by each of the plurality of sample subjects is used as an objective variable. It should be noted here that the sample subject need only be the same biological species as the target subject. The sample signal waveform indicates a time-series change in the area of a region of a heart of a sample subject for whom the type of tachyarrhythmia suffered has been specified (diagnosed) in advance by a medical worker such as a doctor. The type information is the type of tachyarrhythmia specified in advance by a medical worker such as a doctor for each of the sample subjects.

Characteristics corresponding to the type of tachyarrhythmia suffered by the sample subject appear in the shape of the sample signal waveform. With the above configuration, it is possible to generate the inference model 1051 that has machine-learned a relationship between the type of tachyarrhythmia occurring in the heart of the sample subject and the shape of the input waveform generated using the sample signal waveform generated from the image of the heart. By using the trained inference model 1051, it is possible to accurately infer the type of tachyarrhythmia suffered by the target subject from the shape of the target signal waveform based on the image obtained by image-capturing of the heart of the target subject.

Processing in which the inference section 105 infers the type of tachyarrhythmia suffered by the target subject from the target signal waveform using the inference model 1051 will be described with reference to FIG. 4. FIG. 4 is a diagram for explaining an example of processing performed by the inference section 105.

A target signal waveform RA1 shown in the upper part of FIG. 4 indicates a time-series change in the area of the region of a right atrium of a heart of a certain target subject. This target signal waveform RA1 beats 10 times in 3 seconds and has a frequency in a frequency band (approximately 200 bpm) higher than a reference frequency band predetermined as a normal range. This target subject suffers from tachyarrhythmia. When the obtaining section 101 obtains the target signal waveform RA1, the inference section 105 inputs the target signal waveform RA1 to the inference model 1051, thereby outputting "AFL", which is the type of tachyarrhythmia suffered by the target subject, from the inference model 1051.

Meanwhile, a target signal waveform RA2 shown in the lower part of FIG. 4 indicates a time-series change in the area of a region of a right atrium of a heart of another target subject. This target signal waveform RA2 beats 9 times in 3 seconds and has a frequency in a frequency band (approximately 180 bpm) higher than the reference frequency band predetermined as the normal range. This target subject suffers from tachyarrhythmia. When the obtaining section 101 obtains the target signal waveform RA2, the inference section 105 inputs the target signal waveform RA2 to the inference model 1051, thereby outputting "SVT", which is the type of tachyarrhythmia suffered by the target subject, from the inference model 1051.

The inference section 105 may be configured to cut out a waveform corresponding to one or several heartbeats of the target subject from the target signal waveforms RA1 and RA2 and input the cut-out waveform to the inference model 1051. Alternatively, the inference section 105 may be configured to cut out a waveform for a predetermined time from the target signal waveforms RA1 and RA2 and input the cut-out waveform to the inference model 1051. In the example shown in FIG. 4, waveforms for 3 seconds cut out from the target signal waveforms RA1 and RA2 are input to the inference model 1051.

Although FIG. 4 shows a case where the target signal waveforms RA1 and RA2 indicating the time-series change in the area of the region of the right atrium of the target subject in the input image are used, the present invention is not limited to this configuration. That is, the inference section 105 may infer the type of tachyarrhythmia suffered by the target subject by using a target signal waveform indicating a time-series change in the area of any one of the respective regions of the right atrium, the left atrium, the right ventricle, and the left ventricle of the target subject in the input image.

Returning to FIG. 3, the output control section 106 causes various output devices to output the inference result by the inference section 105. For example, when the display device 4 is connected via the output IF section 14, the output control section 106 may cause the display device 4 to display the inference result. It should be noted that any mode of outputting the inference result may be employed, and the output control section 106 may output the inference result by display output, audio output, print output, a combination thereof, or the like.

### Processing performed by arrhythmia type inference device 1

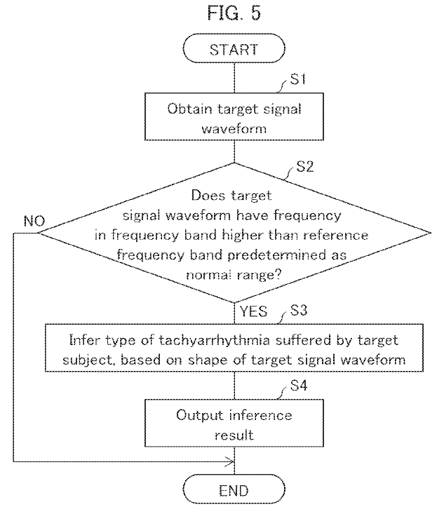

Next, processing (arrhythmia type inference method) performed by the arrhythmia type inference device 1 will be described with reference to FIG. 5. FIG. 5 is a flowchart showing an example of a flow of processing performed by the arrhythmia type inference device 1.

First, the obtaining section 101 obtains the target signal waveform (Step S1: obtaining step).

Next, when the target signal waveform obtained by the obtaining section 101 has a frequency in a frequency band higher than the reference frequency band predetermined as the normal range (YES in Step S2), the inference section 105 infers the type of tachyarrhythmia suffered by the target subject based on the shape of the target signal waveform (Step S3: inference step). Then, the output control section 106 causes the output device (for example, the display device 4) to output the inference result by the inference section 105 (Step S4: output step).

Meanwhile, when the target signal waveform obtained by the obtaining section 101 does not have a frequency in the frequency band higher than the reference frequency band (NO in Step S2), the inference of the type of tachyarrhythmia by the inference section 105 is not performed.

With the above configuration, the arrhythmia type inference device 1 can accurately infer the type of tachyarrhythmia suffered by the target subject, from the image obtained by image-capturing of the heart of the target subject. For example, the arrhythmia type inference device 1 can infer with a high accuracy rate whether the type of tachyarrhythmia of the fetus is "AFL" or "SVT".

### Embodiment 2

Another embodiment of the present invention will be discussed below. For convenience of description, members having the same functions as the members described in the above embodiment are denoted by the same reference numerals, and description thereof will not be repeated.

The arrhythmia type inference device 1 according to the above embodiment is capable of inferring the type of tachyarrhythmia suffered by the target subject using the target signal waveform as it is. Meanwhile, an arrhythmia type inference device 1a according to the present embodiment is configured to infer the type of tachyarrhythmia suffered by the target subject by using one or more target single-beat waveforms corresponding to respective single heartbeats of the heart of the target subject from the target signal waveform.

Similarly to the above embodiment, the arrhythmia type inference device 1a having a function of inferring the type of tachyarrhythmia suffered by the target subject from the target signal waveform obtained from the image analysis device 3 will be described below as an example. However, the arrhythmia type inference device 1a may also have the function of the image analysis device 3. In this case, the arrhythmia type inference device 1a can generate the target signal waveform from the input image obtained from the image capturing device 2 and infer the type of tachyarrhythmia suffered by the target subject.

### Configuration of arrhythmia type interference device 1a

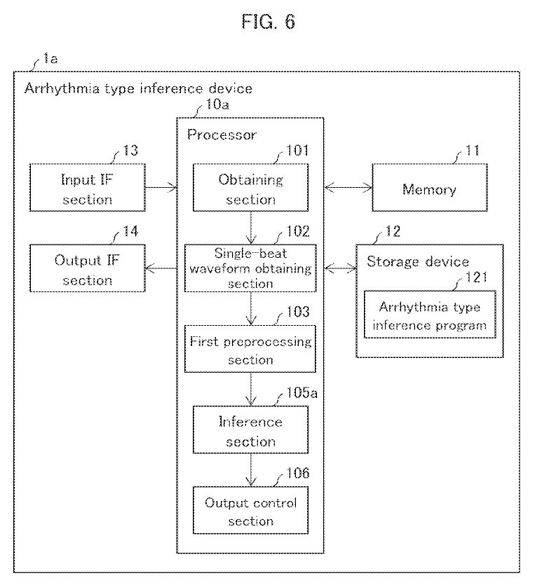

The configuration of the arrhythmia type inference device 1a according to an embodiment of the present invention will be described with reference to FIG. 6. FIG. 6 is a block diagram showing an example of the configuration of the main parts of the arrhythmia type inference device 1a.

As illustrated, the arrhythmia type inference device 1a includes a processor 10a, the memory 11, and the storage device 12. The arrhythmia type inference device 1a may be a personal computer, a server, or a workstation. The processor 10a functions as each section from the obtaining section 101 to the output control section 106 described later by loading the arrhythmia type inference program 121 stored in the storage device 12 into the memory 11 and executing the program.

The processor 10a can be achieved by a logic circuit (hardware) formed in an integrated circuit (IC chip) or the like, or can be achieved by software. When achieved by software, the processor 10a may be composed of, for example, a CPU, a GPU, or a combination thereof. In this case, the software is stored in the storage device 12. Then, the processor 10a reads the software into the memory 11 and executes the software.

The processor 10a functions as each of the obtaining section 101 that obtains the target signal waveform from the image analysis device 3, a single-beat waveform obtaining section 102, a first preprocessing section 103, an inference section 105a, and the output control section 106 by executing the arrhythmia type inference program 121.

The single-beat waveform obtaining section 102 obtains, from the target signal waveform obtained by the obtaining section 101, one or more target single-beat waveforms corresponding to respective single heartbeats of the heart of the target subject.

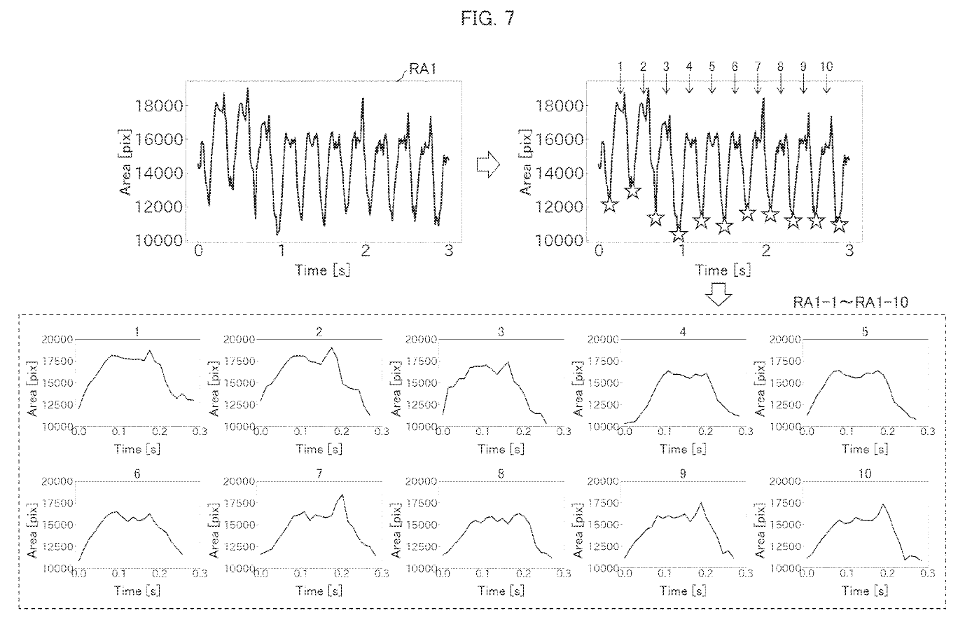

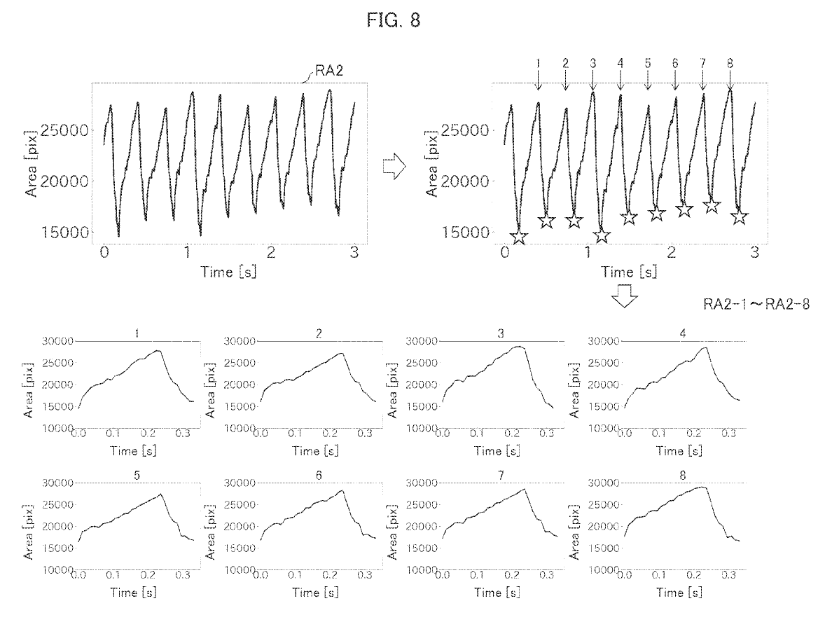

It should be noted here that processing in which the single-beat waveform obtaining section 102 obtains one or more target single-beat waveforms from the target signal waveform will be described with reference to FIGS. 7 and 8. FIGS. 7 and 8 are diagrams for explaining an example of processing performed by the single-beat waveform obtaining section 102.

When the obtaining section 101 obtains the target signal waveform RA1, the single-beat waveform obtaining section 102 first analyzes a pattern of area change accompanying the pulsation of the heart of the target subject in the target signal waveform RA1, and detects one or more time points serving as delimiters of respective pulsations of the heart of the target subject. For example, a diagram shown in an upper right of FIG. 7 shows a state in which the single-beat waveform obtaining section 102 detects, from the target signal waveform RA1, a time point (star mark in the diagram) at which the heart turns from a systole to a diastole in the target signal waveform RA1 as a delimiter of each pulsation of the heart of the target subject. Subsequently, the single-beat waveform obtaining section 102 obtains target single-beat waveforms RA1-1 to RA1-10 cut out from the target signal waveform RA1 based on the detected delimiters. FIG. 7 shows each of the ten target single-beat waveforms obtained from the target signal waveform RA1.

When the obtaining section 101 obtains the target signal waveform RA2, the single-beat waveform obtaining section 102 first analyzes a pattern of area change accompanying the pulsation of the heart of the target subject in the target signal waveform RA2, and detects one or more time points serving as delimiters of respective pulsations of the heart of the target subject. For example, a diagram shown in an upper right of FIG. 8 shows a state in which the single-beat waveform obtaining section 102 detects, from the target signal waveform RA2, a time point (star mark in the diagram) at which the heart turns from a systole to a diastole in the target signal waveform RA2 as a delimiter of each pulsation of the heart of the target subject. Subsequently, the single-beat waveform obtaining section 102 obtains target single-beat waveforms RA2-1 to RA2-8 cut out from the target signal waveform RA2 based on the detected time points serving as delimiters. FIG. 8 shows each of the eight target single-beat waveforms obtained from the target signal waveform RA2.

FIGS. 7 and 8 show an example in which the single-beat waveform obtaining section 102 detects the time point (star mark in the diagram) at which the heart turns from the systole to the diastole in order to obtain the target single-beat waveform. However, the single-beat waveform obtaining section 102 may be capable of detecting a given time point serving as a delimiter of each pulsation of the heart of the target subject in the target signal waveform in order to obtain the target single-beat waveform.

For example, the single-beat waveform obtaining section 102 may be configured to detect a time point at which the area of the region of the heart becomes a predetermined value (for example, 14,000 pix) in the diastole in the target signal waveform. In this case, the single-beat waveform obtaining section 102 obtains, as the target single-beat waveform, a waveform from a time point at which the area of the region of the heart becomes the predetermined value in the diastole to a time point at which the area of the region of the heart becomes the predetermined value in the next diastole.

The single-beat waveform obtaining section 102 may use a trained model such as a convolutional neural network in order to detect the time point serving as the delimiter of each pulsation of the heart of the target subject from the target signal waveform. Such a trained model is constructed by, for example, machine learning using learning data in which a sample signal waveform of a sample subject is used as an explanatory variable and a time point serving as a delimiter of each pulsation of the heart in the sample signal waveform is used as an objective variable.

Furthermore, FIGS. 7 and 8 show a case where the single-beat waveform obtaining section 102 obtains the target single-beat waveform from the target signal waveforms RA1 and RA2 indicating the time-series change in the area of the region of the right atrium of the target subject in the input image. However, the present invention is not limited to this configuration. That is, the single-beat waveform obtaining section 102 can obtain the target single-beat waveform from a target signal waveform indicating a time-series change in the area of any one of the respective regions of the right atrium, the left atrium, the right ventricle, and the left ventricle of the target subject in the input image.

Returning to FIG. 6, the first preprocessing section 103 performs a first preprocessing including at least one of standardization of an amplitude indicating a magnitude of a change in the area of the region of the heart and normalization of a time length of the single heartbeat of the heart, which are shown in each of the one or more target single-beat waveforms. It should be noted here that the standardization of the amplitude is processing of calculating an average value and a standard deviation of an area data group in each of the target single-beat waveforms and dividing the difference between a numerical value of each area data group and the average value by the standard deviation. By this standardization, the area data group in each of the target single-beat waveforms has an average of 0 and a standard deviation of 1, so that it is possible to remove influences due to the difference in the area of the region (that is, the difference in the size of the heart) and the difference in the magnitude (amplitude) of the area change in the target single-beat waveform. Meanwhile, the normalization of the time length of the single heartbeat is processing of aligning widths in a time axis direction of the respective target single-beat waveforms.

Since there are individual differences in the size of the heart and the time of the single heartbeat, the area and the time width in the target single-beat waveform obtained from the target signal waveform differ for each target subject. Therefore, the first preprocessing section 103 performs the first preprocessing on each of the one or more target single-beat waveforms.

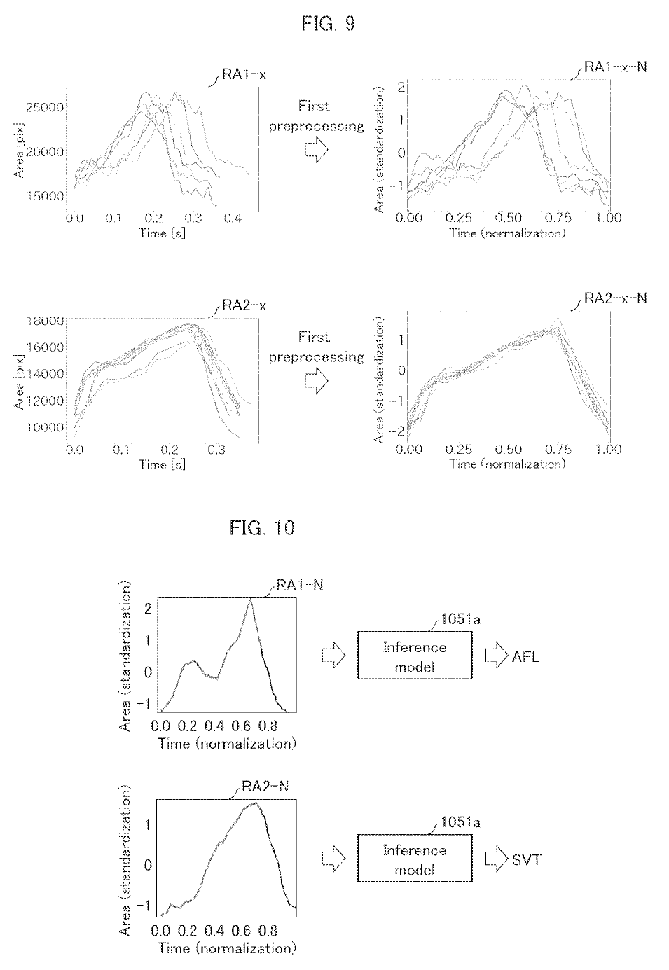

The first preprocessing performed by the first preprocessing section 103 will be described with reference to FIG. 9. FIG. 9 is a diagram for explaining an example of the first preprocessing. FIG. 9 takes, as an example, a case where the first preprocessing section 103 performs the standardization of the amplitude and the normalization of the time length of the single heartbeat.

The upper part of FIG. 9 shows target single-beat waveforms RA1-x-N after the first preprocessing is performed on each of a plurality of target single-beat waveforms RA1-x obtained from the target signal waveform RA1. Meanwhile, the lower part of FIG. 9 shows target single-beat waveforms RA2-x-N after the first preprocessing is performed on each of a plurality of target single-beat waveforms RA2-x obtained from the target signal waveform RA2. As shown in FIG. 9, the shape of the target single-beat waveform RA1-x-N has the same characteristics as the shape of the target single-beat waveform RA1-x before the first preprocessing is performed, and the shape of the target single-beat waveform RA2-x-N has the same characteristics as the shape of the target single-beat waveform RA2-x before the first preprocessing is performed.

Returning to FIG. 6, the inference section 105a infers the type of tachyarrhythmia suffered by the target subject based on the shape of the one or more target single-beat waveforms after the first preprocessing is performed. It should be noted here that the inference section 105a may, using a trained inference model 1051a, infer the type of tachyarrhythmia suffered by the target subject from the one or more target single-beat waveforms after the first preprocessing is performed. In this case, an input waveform used as an explanatory variable in machine learning for generating the inference model 1051a may be generated by performing a third preprocessing on each of one or more sample single-beat waveforms corresponding to respective single heartbeats of the heart of the sample subject in the sample signal waveform. The third preprocessing is the same processing as the first preprocessing performed on each of one or more sample single-beat waveforms. Specifically, the third preprocessing is processing including at least one of standardization of an amplitude indicating a magnitude of a change in the area of the region of the heart and normalization of a time length of the single heartbeat of the heart, for each of the one or more sample single-beat waveforms.

With the above configuration, it is possible to generate the inference model 1051a that has machine-learned a relationship between the type of tachyarrhythmia occurring in the heart of the sample subject and the shape of the sample single-beat waveform generated by using the sample signal waveform generated from the image of the heart. By using the trained inference model 1051a, it is possible to accurately infer the type of tachyarrhythmia suffered by the target subject from the shape of the target single-beat waveform generated from the target signal waveform based on the image obtained by image-capturing of the heart of the target subject.

Processing in which the inference section 105a infers the type of tachyarrhythmia suffered by the target subject from the target single-beat waveform after the first preprocessing is performed using the inference model 1051a will be described with reference to FIG. 10. FIG. 10 is a diagram for explaining an example of processing performed by the inference section 105a.

The upper part of FIG. 10 shows the target single-beat waveform RA1-N after the first preprocessing is performed, obtained from the target signal waveform RA1 indicating the time-series change in the area of the region of the right atrium of the heart of the target subject suffering from tachyarrhythmia. The inference section 105a inputs the target single-beat waveform RA1-N after the first preprocessing is executed into the inference model 1051a, thereby outputting "AFL", which is the type of tachyarrhythmia suffered by the target subject, from the inference model 1051a.

Meanwhile, the lower part of FIG. 10 shows the target single-beat waveform RA2-N after the first preprocessing is executed, obtained from the target signal waveform RA2 indicating the time-series change in the area of the region of the right atrium of the heart of another target subject suffering from tachyarrhythmia. The inference section 105a inputs the target single-beat waveform RA2-N after the first preprocessing is executed to the inference model 1051a, thereby outputting "SVT", which is the type of tachyarrhythmia suffered by the target subject, from the inference model 1051a.

Although FIG. 10 shows a configuration in which one target single-beat waveform is input into the inference model 1051a and one inference result is output, the inference section 105a is not limited to this configuration. The inference section 105a may be configured to infer the type of tachyarrhythmia suffered by the target subject based on one or more inference results output from the inference model 1051a into which each of the one or more target single-beat waveforms after the first preprocessing is performed is input.

For example, the inference section 105a may input each of six target single-beat waveforms shown in the target single-beat waveforms RA1-x-N shown in FIG. 9 into the inference model 1051a. In this case, if a predetermined ratio (for example, 60%) or more of six inference results output from the inference model 1051a is "AFL", the inference section 105a may infer that the type of tachyarrhythmia suffered by the target subject is "AFL". Similarly, for example, the inference section 105a may input each of eight target single-beat waveforms shown in the target single-beat waveforms RA2-x-N shown in FIG. 9 into the inference model 1051a. In this case, if a predetermined ratio (for example, 60%) or more of eight inference results output from the inference model 1051a is "SVT", the inference section 105a may infer that the type of tachyarrhythmia suffered by the target subject is "SVT".

### Processing performed by arrhythmia type inference device 1a

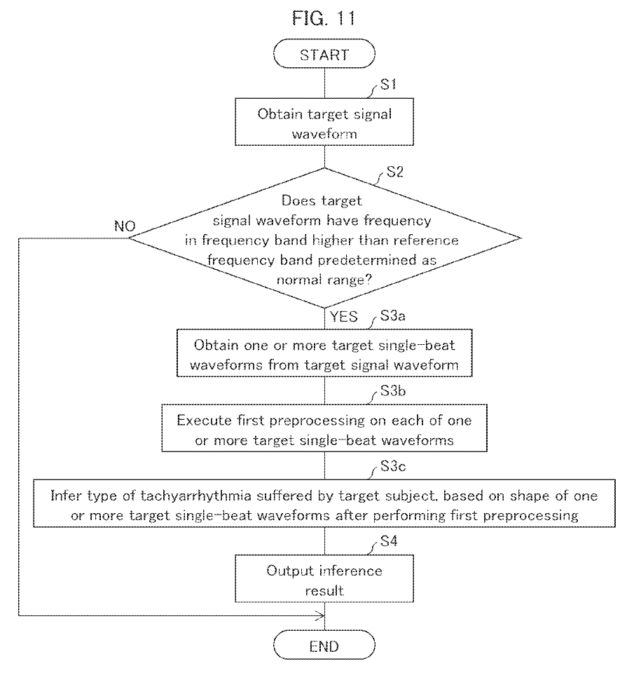

Next, processing (arrhythmia type inference method) performed by the arrhythmia type inference device 1a will be described with reference to FIG. 11. FIG. 11 is a flowchart showing an example of a flow of processing performed by the arrhythmia type inference device 1a.

First, the obtaining section 101 obtains the target signal waveform (Step S1: obtaining step).

Next, when the target signal waveform obtained by the obtaining section 101 has a frequency in a frequency band higher than the reference frequency band predetermined as the normal range (YES in Step S2), the single-beat waveform obtaining section 102 obtains one or more target single-beat waveforms from the target signal waveform (Step S3a: single-beat waveform obtaining step).

Subsequently, the first preprocessing section 103 executes the first preprocessing on each of the obtained one or more target single-beat waveforms (Step S3b: first preprocessing step).

Next, the inference section 105a infers the type of tachyarrhythmia suffered by the target subject based on the shape of the target single-beat waveform after the first preprocessing is performed (Step S3c: inference step). Then, the output control section 106 causes the output device (for example, the display device 4) to output the inference result by the inference section 105a (Step S4: output step).

With the above configuration, the arrhythmia type inference device 1a can more accurately infer the type of tachyarrhythmia suffered by the target subject from the image obtained by image-capturing of the heart of the target subject.

### Embodiment 3

Another embodiment of the present invention will be discussed below. For convenience of description, members having the same functions as the members described in the above embodiment are denoted by the same reference numerals, and description thereof will not be repeated.

Similarly to the above embodiment, the arrhythmia type inference device 1b having a function of inferring the type of tachyarrhythmia suffered by the target subject from the target signal waveform obtained from the image analysis device 3 will be described below as an example. However, the arrhythmia type inference device 1b may also have the function of the image analysis device 3. In this case, the arrhythmia type inference device 1b generates the target signal waveform from the input image obtained from the image capturing device 2 and infers the type of tachyarrhythmia suffered by the target subject.

### Configuration of arrhythmia type inference device 1b

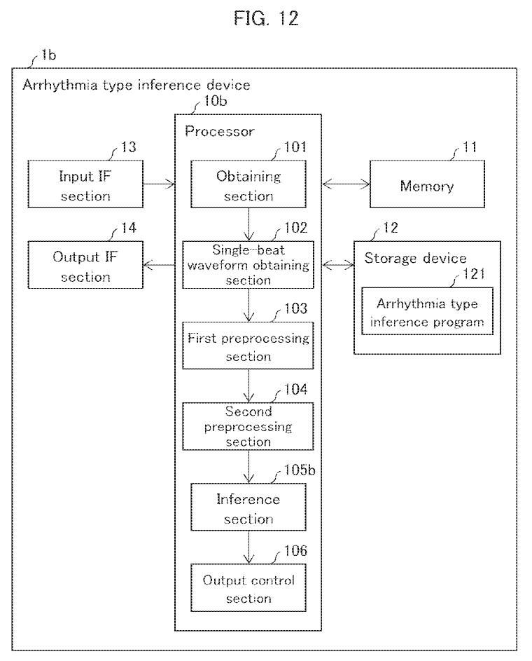

The configuration of the arrhythmia type inference device 1b according to an embodiment of the present invention will be described with reference to FIG. 12. FIG. 12 is a block diagram showing an example of the configuration of the main parts of the arrhythmia type inference device 1b.

As illustrated, the arrhythmia type inference device 1b includes a processor 10b, the memory 11, and the storage device 12. The arrhythmia type inference device 1b may be a personal computer, a server, or a workstation. The processor 10b functions as each section from the obtaining section 101 to the output control section 106 described later by loading the arrhythmia type inference program 121 stored in the storage device 12 into the memory 11 and executing the program.

The processor 10b can be achieved by a logic circuit (hardware) formed in an integrated circuit (IC chip) or the like, or can be achieved by software. When achieved by software, the processor 10b may be composed of, for example, a CPU, a GPU, or a combination thereof. In this case, the software is stored in the storage device 12. Then, the processor 10b reads the software into the memory 11 and executes the software.

The processor 10b functions as each of the obtaining section 101 that obtains the target signal waveform from the image analysis device 3, the single-beat waveform obtaining section 102, the first preprocessing section 103, a second preprocessing section 104, an inference section 105b, and the output control section 106 by executing the arrhythmia type inference program 121.

The second preprocessing section 104 performs a second preprocessing of extracting a part corresponding to a predetermined period of interest in the target single-beat waveform as a target partial waveform, for each of the one or more target single-beat waveforms. That is, the second preprocessing section 104 generates the target partial waveform by performing the second preprocessing on each of the one or more target single-beat waveforms.

The inference section 105b infers the type of tachyarrhythmia suffered by the target subject based on the shape of one or more target partial waveforms. It should be noted here that the inference section 105b may, using a trained inference model 1051b, infer the type of tachyarrhythmia suffered by the target subject from the one or more target partial waveforms. In this case, an input waveform used as an explanatory variable in machine learning for generating the inference model 1051b may be generated by performing the third preprocessing and a fourth preprocessing on each of one or more sample single-beat waveforms corresponding to respective single heartbeats of the heart of the sample subject in the sample signal waveform. The fourth preprocessing is processing of extracting a part corresponding to a predetermined period of interest in the sample single-beat waveform from each of the one or more sample single-beat waveforms.

For example, when the type of tachyarrhythmia is "AFL", there is a characteristic in the shape of the target single-beat waveform RA1-N in a period of 0 or more and 0.6 to 0.8 or less on a horizontal axis indicating time after normalization, which is significantly different from a case where the type of tachyarrhythmia is "SVT". Meanwhile, in the target single-beat waveform RA1-N in a period corresponding to 0.6 to 0.8 or more on the horizontal axis indicating the time after normalization, a difference from the case where the type of tachyarrhythmia is "SVT" is small. Therefore, a target partial waveform, which is a part corresponding to a period of interest having a characteristic shape for each type of tachyarrhythmia, may be extracted from the target single-beat waveform, and the type of tachyarrhythmia may be inferred using the target partial waveform.

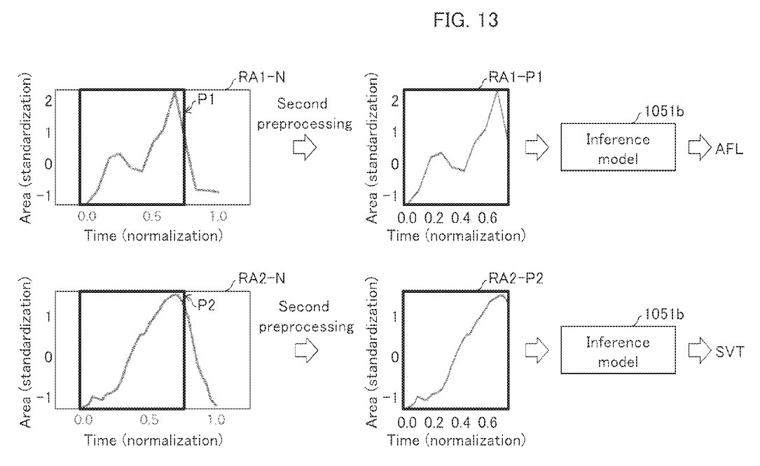

Processing in which the second preprocessing section 104 extracts the target partial waveform from the target single-beat waveform and processing in which the inference section 105b infers the type of tachyarrhythmia suffered by the target subject from the target partial waveform will be described with reference to FIG. 13. FIG. 13 is a diagram for explaining an example of processing performed by the second preprocessing section 104 and the inference section 105b.

The target single-beat waveform RA1-N shown in the upper part of FIG. 13 indicates a time-series change in the area of a region of a right atrium of a heart of a certain target subject. The second preprocessing section 104 extracts a part corresponding to a predetermined period of interest P1 from the target single-beat waveform RA1-N. The inference section 105b inputs a target partial waveform RA1-P1 extracted by the second preprocessing section 104 to the inference model 1051b, and infers that the type of tachyarrhythmia suffered by the target subject is "AFL".

The target single-beat waveform RA2-N shown in the lower part of FIG. 13 indicates a time-series change in the area of a region of a right atrium of a heart of another target subject. The second preprocessing section 104 extracts a part corresponding to a predetermined period of interest P2 from the target single-beat waveform RA2-N. The inference section 105b inputs a target partial waveform RA2-P2 extracted by the second preprocessing section 104 into the inference model 1051b, and infers that the type of tachyarrhythmia suffered by the target subject is "SVT".

FIG. 13 shows, as an example, a case where the target partial waveforms RA1-P1 and RA2-P2 in the periods of interest P1 and P2 corresponding to 0 or more and 0.75 or less on the horizontal axis indicating the time after normalization are used. However, the present invention is not limited to this configuration, and the periods of interest P1 and P2 can be set to any periods in which a difference in waveform depending on the type of tachyarrhythmia suffered by the target subject is remarkable.

Although FIG. 13 shows a configuration in which one target single-beat waveform is input into the inference model 1051b and one inference result is output, the inference section 105b is not limited to this configuration. The inference section 105b may be configured to infer the type of tachyarrhythmia suffered by the target subject based on one or more inference results output from the inference model 1051b into which each of one or more target partial waveforms after the first preprocessing and the second preprocessing are performed with respect to the target signal waveform is input.

For example, if a predetermined ratio (for example, 60%) or more of one or more inference results output from the inference model 1051b is "AFL", the inference section 105b may infer that the type of tachyarrhythmia suffered by the target subject is "AFL". Furthermore, if a predetermined ratio (for example, 60%) or more of one or more inference results output from the inference model 1051b is "SVT", the inference section 105b may infer that the type of tachyarrhythmia suffered by the target subject is "SVT".

### Processing performed by arrhythmia type inference device 1b

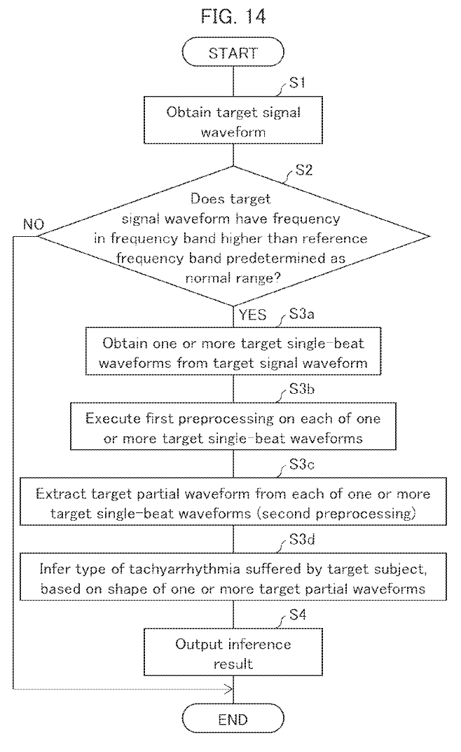

Next, processing (arrhythmia type inference method) performed by the arrhythmia type inference device 1b will be described with reference to FIG. 14. FIG. 14 is a flowchart showing an example of a flow of processing performed by the arrhythmia type inference device 1b.

First, the obtaining section 101 obtains the target signal waveform (Step S1: obtaining step).

Next, when the target signal waveform obtained by the obtaining section 101 has a frequency in a frequency band higher than the reference frequency band predetermined as the normal range (YES in Step S2), the single-beat waveform obtaining section 102 obtains one or more target single-beat waveforms from the target signal waveform (Step S3a: single-beat waveform obtaining step).

Subsequently, the first preprocessing section 103 executes the first preprocessing on each of the obtained one or more target single-beat waveforms (Step S3b: first preprocessing step), and the second preprocessing section 104 extracts the target partial waveform from each of the one or more target single-beat waveforms after the first preprocessing is executed (Step S3c: second preprocessing step).

Next, the inference section 105b infers the type of tachyarrhythmia suffered by the target subject based on the shape of the one or more target partial waveforms (Step S3d: inference step). Then, the output control section 106 causes the output device to output the inference result by the inference section 105b (Step S4: output step).

Meanwhile, if the target signal waveform obtained by the obtaining section 101 does not have a frequency in the frequency band higher than the reference frequency band (NO in Step S2), the inference of the type of tachyarrhythmia by the inference section 105b is not performed.

According to the above configuration, the arrhythmia type inference device 1b extracts the target partial waveform including a characteristic shape corresponding to the type of tachyarrhythmia from each of the one or more target single-beat waveforms after the first preprocessing is performed, and, using the target partial waveform, infers the type of tachyarrhythmia suffered by the target subject. This allows the arrhythmia type inference device 1b to more accurately infer the type of tachyarrhythmia suffered by the target subject.

### Software implementation example

The functions of the arrhythmia type inference devices 1, 1a, and 1b (hereinafter, referred to as a "device") can be realized by a program for causing a computer to function as the device, the program causing the computer to function as the control blocks (particularly, the sections included in the processors 10, 10a, and 10b) of the device.

In this case, the device includes a computer that has at least one control device (for example, a processor) and at least one memory device (for example, a memory) as hardware for executing the program. By the control device executing the program with use of the storage device, the functions described in the foregoing embodiments are realized.

The program can be stored in one or more non-transitory computer-readable storage media. The storage medium can be provided in the device, or the storage medium does not need to be provided in the device. In the latter case, the program can be supplied to or made available to the device via any wired or wireless transmission medium.

Alternatively, a part or all of the functions of the control blocks can be realized by a logic circuit. For example, the present invention encompasses, in its scope, an integrated circuit in which a logic circuit that functions as each of the control blocks is formed. In addition, the function of each of the control blocks can be realized by, for example, a quantum computer.

Each of the processes described in the foregoing embodiments may be carried out by artificial intelligence (AI). In this case, the AI may be operated by the control device or may be operated by another device (for example, an edge computer and a cloud server).

The present invention is not limited to the embodiments, but can be altered by a skilled person in the art within the scope of the claims. The present invention also encompasses, in its technical scope, any embodiment derived by combining technical means disclosed in differing embodiments.

Aspects of the present invention can also be expressed as follows:

An arrhythmia type inference device according to Aspect 1 of the present invention includes: an obtaining section that obtains a target signal waveform indicating a time-series change in an area of a region, which is at least one of a left atrium, a left ventricle, a right atrium, and a right ventricle, the target signal waveform being generated by analyzing an image of a heart of a target subject; and an inference section that infers the type of tachyarrhythmia suffered by the target subject, based on a shape of the target signal waveform having a frequency in a frequency band higher than a reference frequency band predetermined as a normal range.

The arrhythmia type inference device according to Aspect 2 of the present invention, in Aspect 1, may be configured to further include: a single-beat waveform obtaining section that obtains, from the target signal waveform, one or more target single-beat waveforms corresponding to respective single heartbeats of the heart of the target subject; and a first preprocessing section that performs a first preprocessing including at least one of standardization of an amplitude indicating a magnitude of a change in the area of the region and normalization of a time length of the single heartbeat of the heart, which are shown in each of the one or more target single-beat waveforms, wherein the inference section infers the type of the tachyarrhythmia suffered by the target subject, based on a shape of the one or more target single-beat waveforms after the first preprocessing is performed.

The arrhythmia type inference device according to Aspect 3 of the present invention, in Aspect 1 or 2, may be configured to further include a second preprocessing section that performs a second preprocessing of extracting, for each of the one or more target single-beat waveforms, a part of a predetermined period of interest in each of the one or more target single-beat waveforms as a target partial waveform, wherein the target partial waveform includes one or more target partial waveforms, and the inference section infers the type of the tachyarrhythmia suffered by the target subject, based on a shape of the one or more target partial waveforms.

The arrhythmia type inference device according to Aspect 4 of the present invention, in any one of Aspects 1 to 3, may be configured such that the inference section infers the type of the tachyarrhythmia suffered by the target subject from the target signal waveform by using an inference model that has been machine-learned using training data in which an input waveform generated using a sample signal waveform indicating a time-series change in the area of the region generated by analyzing images of hearts of a plurality of sample subjects suffering from tachyarrhythmia is used as an explanatory variable and in which type information indicating the type of the tachyarrhythmia suffered by each of the plurality of sample subjects is used as an objective variable.

The arrhythmia type inference device according to Aspect 5 of the present invention, in Aspect 4, may be configured such that: the input waveform is generated by performing a third preprocessing on each of one or more sample single-beat waveforms corresponding to respective single heartbeats of the heart of each of the plurality of sample subjects in the sample signal waveform, the third preprocessing including at least one of standardization of an amplitude indicating a magnitude of a change in the area of the region and normalization of a time length of the single heartbeat of the heart; and the inference section infers, using the inference model, the type of the tachyarrhythmia suffered by the target subject from the one or more target single-beat waveforms which correspond to the respective single heartbeats of the heart of the target subject obtained from the target signal waveform and which have been subjected to a preprocessing identical to the third preprocessing.

The arrhythmia type inference device according to Aspect 6 of the present invention, in Aspect 5, may be configured such that the inference section infers the type of the tachyarrhythmia suffered by the target subject, based on one or more inference results output from the inference model to which each of the one or more target single-beat waveforms is input after the preprocessing identical to the third preprocessing has been performed with respect to the target signal waveform.

The arrhythmia type inference device according to Aspect 7 of the present invention, in Aspect 4, may be configured such that: the input waveform is generated by performing a third preprocessing on each of one or more sample single-beat waveforms corresponding to the respective single heartbeats of the heart of each of the plurality of sample subjects in the sample signal waveform, the third preprocessing including at least one of standardization of an amplitude indicating a magnitude of a change in the area of the region and normalization of a time length of the single heartbeat of the heart and a fourth preprocessing of extracting a part of a predetermined period of interest in the one or more sample single-beat waveforms from each of the one or more sample single-beat waveforms; and the inference section, using the inference model, infers the type of the tachyarrhythmia suffered by the target subject from one or more target partial waveforms generated by, for each of the one or more target single-beat waveforms, performing a first preprocessing including at least one of standardization of an amplitude indicating a magnitude of a change in the area of the region and normalization of a time length of the single heartbeat of the heart, which are shown in each of the one or more target single-beat waveforms, and a second preprocessing of extracting a part of a predetermined period of interest in each of the one or more target single-beat waveforms as each of the one or more target partial waveforms.

The arrhythmia type inference device according to Aspect 8 of the present invention, in Aspect 7, may be configured such that the inference section infers the type of the tachyarrhythmia suffered by the target subject, based on one or more inference results output from the inference model to which each of the one or more target partial waveforms is input after the first preprocessing and the second preprocessing have been performed with respect to the target signal waveform.

The arrhythmia type inference device according to Aspect 9 of the present invention, in any one of Aspects 1 to 8, may be configured such that: the region is at least one of the left atrium and the right atrium; and the inference section infers whether the type of the tachyarrhythmia suffered by the target subject is supraventricular tachycardia or atrial flutter, based on the shape of the target signal waveform having the frequency in the frequency band higher than the reference frequency band predetermined as the normal range.

An arrhythmia type inference method according to Aspect 10 of the present invention is an arrhythmia type inference method performed by one or more information processing devices, said method including: an obtaining step of obtaining a target signal waveform indicating a time-series change in an area of a region, which is at least one of a left atrium, a left ventricle, a right atrium, and a right ventricle, the target signal waveform being generated by analyzing an image of a heart of a target subject; and an inference step of inferring the type of tachyarrhythmia suffered by the target subject, based on a shape of the target signal waveform having a frequency in a frequency band higher than a reference frequency band predetermined as a normal range.

A non-transitory computer-readable recording medium according to Aspect 11 of the present invention is a recording medium recording an arrhythmia type inference program for causing a computer to function as the arrhythmia type inference device according to any one of Aspects 1 to 9, the arrhythmia type inference program causing the computer to function as the obtaining section and the inference section.

### REFERENCE SIGNS LIST

1, 1a, 1b Arrhythmia Type Inference Device

10, 10a, 10b Processor

101 Obtaining Section

102 Single-Beat Waveform Obtaining Section

103 First Preprocessing Section

104 Second Preprocessing Section

105, 105a, 105b Inference Section

121 Arrhythmia Type Inference Program

1051, 1051a, 1051b Inference Model

1. An arrhythmia type inference device comprising:
an obtaining section that obtains a target signal waveform indicating a time-series change in an area of a region, which is at least one of a left atrium, a left ventricle, a right atrium, and a right ventricle, the target signal waveform being generated by analyzing an image of a heart of a target subject; and
an inference section that infers the type of tachyarrhythmia suffered by the target subject, based on a shape of the target signal waveform having a frequency in a frequency band higher than a reference frequency band predetermined as a normal range.

2. The arrhythmia type inference device according to claim 1, further comprising:
a single-beat waveform obtaining section that obtains, from the target signal waveform, one or more target single-beat waveforms corresponding to respective single heartbeats of the heart of the target subject; and
a first preprocessing section that performs a first preprocessing including at least one of standardization of an amplitude indicating a magnitude of a change in the area of the region and normalization of a time length of the single heartbeat of the heart, which are shown in each of the one or more target single-beat waveforms, wherein
the inference section
infers the type of the tachyarrhythmia suffered by the target subject, based on a shape of the one or more target single-beat waveforms after the first preprocessing is performed.

3. The arrhythmia type inference device according to claim 2, further comprising
a second preprocessing section that performs a second preprocessing of extracting, for each of the one or more target single-beat waveforms, a part of a predetermined period of interest in each of the one or more target single-beat waveforms as a target partial waveform, wherein
the target partial waveform includes one or more target partial waveforms, and the inference section
infers the type of the tachyarrhythmia suffered by the target subject, based on a shape of the one or more target partial waveforms.

4. The arrhythmia type inference device according to claim 1, wherein
the inference section
infers the type of the tachyarrhythmia suffered by the target subject from the target signal waveform by using an inference model that has been machine-learned using training data in which an input waveform generated using a sample signal waveform indicating a time-series change in the area of the region generated by analyzing images of hearts of a plurality of sample subjects suffering from tachyarrhythmia is used as an explanatory variable and in which type information indicating the type of the tachyarrhythmia suffered by each of the plurality of sample subjects is used as an objective variable.

5. The arrhythmia type inference device according to claim 4, wherein:
the input waveform is generated by performing a third preprocessing on each of one or more sample single-beat waveforms corresponding to respective single heartbeats of the heart of each of the plurality of sample subjects in the sample signal waveform, the third preprocessing including at least one of standardization of an amplitude indicating a magnitude of a change in the area of the region and normalization of a time length of the single heartbeat of the heart; and
the inference section
infers, using the inference model, the type of the tachyarrhythmia suffered by the target subject from the one or more target single-beat waveforms which correspond to the respective single heartbeats of the heart of the target subject obtained from the target signal waveform and which have been subjected to a preprocessing identical to the third preprocessing.

6. The arrhythmia type inference device according to claim 5, wherein
the inference section
infers the type of the tachyarrhythmia suffered by the target subject, based on one or more inference results output from the inference model to which each of the one or more target single-beat waveforms is input after the preprocessing identical to the third preprocessing has been performed with respect to the target signal waveform.

7. The arrhythmia type inference device according to claim 4, wherein:
the input waveform is generated by performing
a third preprocessing on each of one or more sample single-beat waveforms corresponding to the respective single heartbeats of the heart of each of the plurality of sample subjects in the sample signal waveform, the third preprocessing including at least one of standardization of an amplitude indicating a magnitude of a change in the area of the region and normalization of a time length of the single heartbeat of the heart and
a fourth preprocessing of extracting a part of a predetermined period of interest in the one or more sample single-beat waveforms from each of the one or more sample single-beat waveforms; and
the inference section,
using the inference model, infers the type of the tachyarrhythmia suffered by the target subject from one or more target partial waveforms generated by, for each of the one or more target single-beat waveforms, performing
a first preprocessing including at least one of standardization of an amplitude indicating a magnitude of a change in the area of the region and normalization of a time length of the single heartbeat of the heart, which are shown in each of the one or more target single-beat waveforms, and
a second preprocessing of extracting a part of a predetermined period of interest in each of the one or more target single-beat waveforms as each of the one or more target partial waveforms.

8. The arrhythmia type inference device according to claim 7, wherein
the inference section
infers the type of the tachyarrhythmia suffered by the target subject, based on one or more inference results output from the inference model to which each of the one or more target partial waveforms is input after the first preprocessing and the second preprocessing have been performed with respect to the target signal waveform.

9. The arrhythmia type inference device according to claim 1, wherein:
the region is at least one of the left atrium and the right atrium; and
the inference section
infers whether the type of the tachyarrhythmia suffered by the target subject is supraventricular tachycardia or atrial flutter, based on the shape of the target signal waveform having the frequency in the frequency band higher than the reference frequency band predetermined as the normal range.

10. An arrhythmia type inference method performed by one or more information processing devices, said method comprising:
an obtaining step of obtaining a target signal waveform indicating a time-series change in an area of a region, which is at least one of a left atrium, a left ventricle, a right atrium, and a right ventricle, the target signal waveform being generated by analyzing an image of a heart of a target subject; and
an inference step of inferring the type of tachyarrhythmia suffered by the target subject, based on a shape of the target signal waveform having a frequency in a frequency band higher than a reference frequency band predetermined as a normal range.

11. A non-transitory computer-readable recording medium recording an arrhythmia type inference program for causing a computer to function as the arrhythmia type inference device according to claim 1, the arrhythmia type inference program causing the computer to function as the obtaining section and the inference section.
